(19) United States
(12) Patent Application Publication
(10) Pub. No.: US 2008/0220100 A1
(43) Pub. Date: Sep. 11, 2008

(54) Fermented panax notoginseng solution with anti-cancer effects and manufacture method thereof

(75) Inventor: Kung-Ming Lu, Taipei City (TW)

Correspondence Address: BIRCH STEWART KOLASCH & BIRCH, PO BOX 747, FALLS CHURCH, VA, 22040-0747, US

(73) Assignee: MICROBIO COMPANY LTD., TAIWAN

(21) Appl. No.: 11/896,762

(22) Filed: Sep. 5, 2007

(30) Foreign Application Priority Data
Mar. 6, 2007 (TW) 096107639

(51) Int. Cl. A61K 36/258; A61P 35/00; C12P 1/04

(52) U.S. Cl. 424/728; 435/170

(57) ABSTRACT

This invention relates to a fermented Panax notoginseng solution and manufactured method thereof. The manufacture method comprises activating a Lactobacillus spp, fermenting a Panax notoginseng medium with the Lactobacillus spp. to form a fermented solution, and centrifuging the fermented solution to obtain a supernatant. The supernatant has anti-cancer effects.

### BACKGROUND OF THE INVENTION

1. Field of the Invention

The present invention relates to an anti-cancer composition, and in particular relates to a fermented Panax notoginseng solution with anti-cancer effects and manufacture method thereof.

2. Description of the Related Art

Cancer is estimated to be the second most prevalent disease in populations worldwide. Nearly all cancers of the organs are incurable. Presently, treatment of cancer includes surgery, radiation therapy, immunotherapy, chemotherapy, and alcohol injection among others. Most anti-tumor drugs have a higher toxicity to rapidly proliferating cancer cells (such as leukemia, or lymphoma) than to slowly proliferating cancer cells (such as liver cancer, or lung cancer). Many anti-cancer drugs additionally exhibit poor selectivity to cancer cells, resulting in undesirable side effects. A feasible alternative is to explore active ingredients in Chinese herbal medicines as an adjuvant or primary therapy for cancer treatments.

Liver cancer is tumor of the liver and is classified into 2 types. One is primary liver cancer, and 60% of primary liver cancer is developed from liver cirrhosis. Another is metastatic liver cancer, which is transferred from other organs. Liver cancer can be classified into 3 types by tumor form, including (1) block form, (2) node form, and (3) disseminative form, wherein the node form tumor is most prevalent. In addition, liver cancer also can be classified into 4 types by histology, including (1) hepatocellular carcinoma, (2) cholangiocarcinoma, (3) mixed hepatocholangiocacinoma, and (4) hepatoblastoma. Approximately 80% of patients have hepatocellular carcinoma.

In clinic therapy, treatment of liver cancer includes surgical resection or topical treatments, such as percutaneous ethanol injection, radiofrequency ablation, cryotherapy, percutaneous microwave coagulation (Carr, 2004; Labonte et al., 2000). However, efficiency of almost all treatments is less to be desired. For example, the success rate of surgical resection is only 10 to 20%, and 5 years after the surgical operation, the survival rate is just 20 to 50%, while recurrence rate is about 100%. Furthermore, traditional anti-cancer drugs usually have a lot of harmful effects, such as high toxicity, low specificity, and immune inhibition. For example, doxorubicin, a chemotherapeutic drug belonging to the antibiotic family of drugs has side effects, which include cardiomyopathy rate increase, congestive heart failure, and nausea.

Panax notoginseng is a Chinese herb often known by its common name, Tienchi ginseng. Tienchi ginseng has been used in China for centuries. Panax notoginseng has already been investigated as a possible agent to treat cardiovascular disorders, to relieve symptoms of angina pectoris and also to reduce high blood pressure. It may lower serum cholesterol levels as well. Thus, to prevent cancer and protect the liver, a less harmful (side effects) and more effective composition is desirable.

### BRIEF SUMMARY OF INVENTION

The invention provides a method for fermenting Panax notoginseng, comprising activating a Lactobacillus spp., fermenting a Panax notoginseng medium with the Lactobacillus spp. to form a fermented solution, and centrifuging the fermented solution to obtain a supernatant.

The invention further provides a fermented Panax notoginseng solution prepared by the above method, wherein the fermented Panax notoginseng solution suppresses the growth of cancer cells.

The invention further provides a composition of preventing and/or treating cancer, comprising an effective amount of fermented Panax notoginseng solution prepared by the above method, and a pharmaceutically acceptable carrier, or excipient.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIGS. 1a-1b show that the fermented Panax notoginseng medium inhibits the growth of human hepatocellular carcinoma Hep 3B and Hep G2 cells;

FIGS. 2a-2b show the anti-cancer effect of signal and mixed strain lactic acid bacteria fermentation;

FIGS. 3a-3b show the anti-cancer effect of the fermented product at 32° C.;

FIGS. 3c-3d show the anti-cancer effect of the fermented product at 37° C.;

FIGS. 3e-3f show the anti-cancer effect of the fermented product at 42° C.;

FIGS. 4a-4b show the anti-cancer effect of the fermented product at pH 3.5;

FIGS. 4c-4d show the anti-cancer effect of the fermented product at pH 4.5;

FIGS. 4e-4f show the anti-cancer effect of the fermented product at pH 5.5;

FIG. 5 shows that black bean induces anti-oxidation of the fermented product;

FIGS. 6a-6b show that the pre-heating of Panax notoginseng medium induces anti-cancer effect;

FIG. 7 shows the effect of the fermented product on normal liver cells;

FIG. 8 shows that the fermented product inhibits Hep 3B cells having HBsAg, and

FIG. 9 shows that the high dosage fermented product represses tumor growth.

### DETAILED DESCRIPTION OF INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides a method for fermenting Panax notoginseng, comprising activating a Lactobacillus spp, fermenting a Panax notoginseng medium with the Lactobacillus spp. to form a fermented solution, and centrifuging the fermented solution to obtain a supernatant.

In the fermentation method of the invention, first, lactic acid bacteria are activated by a medium. The medium can be any one suitable medium, such as MSR medium or peptone-yeast extract base medium. The lactic acid bacteria can include Streptococcus salivarius subsp. thermophilus (BCRC12268), Lactobacillus helveticus (BCRC14092, Lactobacillus rhamnosus GG.(BCRC16000), Lactobacillus acidophilus (BCRC10695), Bifidobacterium longum (BCRC14602), Bifidobacterium catenulatum (BCRC14667), Bifidobacterium breve (BCRC11846), Bifidobacterium bifidum (BCRC14615), or combinations thereof.

Next, at least one lactic acid bacteria is added to a Panax notoginseng medium. In the invention, single or mixed lactic acid bacteria can be used with mixed lactic acid bacteria preferable. The Panax notoginseng medium of the invention includes 10 to 30 wt % of Panax notoginseng powder and 70 to 90 wt % of water, preferably, 10 to 15 wt % of Panax notoginseng powder and 85 to 90 wt % of water. The Panax notoginseng can be classified into 20, 40, and 60 root warts according to the size of root warts. In the invention, 40 and 60 warts are preferable. The fermentation time is about 1 to 7 days, preferably, 1 to 3. The fermentation temperature is about 25 to 42° C., preferably, 30 to 40° C., more preferably, 35 to 37° C. The pH value of fermentation is about 3.0 to 5.0, preferably, 3.5 to 4.5. It should be noted that the fermented product is inactive when pH values exceeds 5.5. Additionally, the Panax notoginseng medium can be stirred to increase fermentation rate during the fermentation process.

After fermentation, the Panax notoginseng medium is heated to stop the fermentation processing, and then the bacteria and nitrogen carbon compounds are removed by centrifugation to obtain the fermented solution of the invention.

In another embodiment, the fermentation method of the invention further comprises adding other functional materials, such as black bean or bupleurum extract, to the Panax notoginseng medium to increase the bio-activity (such as anti-oxidation) of the fermented solution of the invention.

In another embodiment, the Panax notoginseng medium can be pre-heated to induce anti-cancer effect. The temperature of the pre-heating can exceed 75° C., preferably, 100° C. The time of the pre-heating can exceed 10 min, preferably, 20 to 180 min.

The invention further provides a Panax notoginseng fermented solution. The Panax notoginseng fermented solution can suppress the growth of cancer cells with the suppress rate exceeding 60%. The type of suppressed cancer cells of the invention include breast, prostate, blood, colorectal, uterine, ovarian, endometrial, cervical, testicular, malignant lymphoma, rhabdomyosarcoma, neuroblastoma, pancreatic, lung, brain, skin, gastric, liver, kidney, and nasopharyngeal cancer cells.

Although the Panax notoginseng fermented solution of the invention can suppress cancer cell growth, it does not kill normal cells. The LD₅₀ of the Panax notoginseng fermented solution for primary mouse hepatocyte exceeds 900 μg/ml, preferably, 970 μg/ml.

In addition, the Panax notoginseng fermented solution not only suppresses the growth of cancer cells but also inhibits hepatitis B virus surface antigen (HBsAg). The fermentation time is important, since the anti-HBsAg effect will be increased with time. The inhibition rate of HBsAg can exceed 80%.

Furthermore, the invention further provides a composition for preventing and/or treating cancer, comprising an effective amount of fermented Panax notoginseng solution, and a pharmaceutically acceptable carrier, or excipient. The type of cancer prevented and/or treated include breast, prostate, blood, colorectal, uterine, ovarian, endometrial, cervical, testicular, malignant lymphoma, rhabdomyosarcoma, neuroblastoma, pancreatic, lung, brain, skin, gastric, liver, kidney, and nasopharyngeal cancers. Preferably the composition is used for the prevention and treatment of liver cancer. The suppression rate is 60% or more. The composition can administrate orally or by injection.

The composition of the invention prevents and/or treats cancer so that the composition can be administrated to cancer patients, chemotherapy patients, and high-risk group cancer patients, etc. In addition, the composition is very safe and does not cause biological damage so that the composition can also serve as a food supplement.

### EXAMPLE

### Example 1

### Preparation of Panax notoginseng Medium

First, Panax notoginseng was ground into powder with 120-mesh particle size. Then, 10 g of the Panax notoginseng powder was added to 90 ml of water and sterilized by autoclave for 20 min to form the Panax notoginseng medium of the invention.

### Example 2

### Fermenting Panax notoginseng Medium with Lactic Acid Bacteria

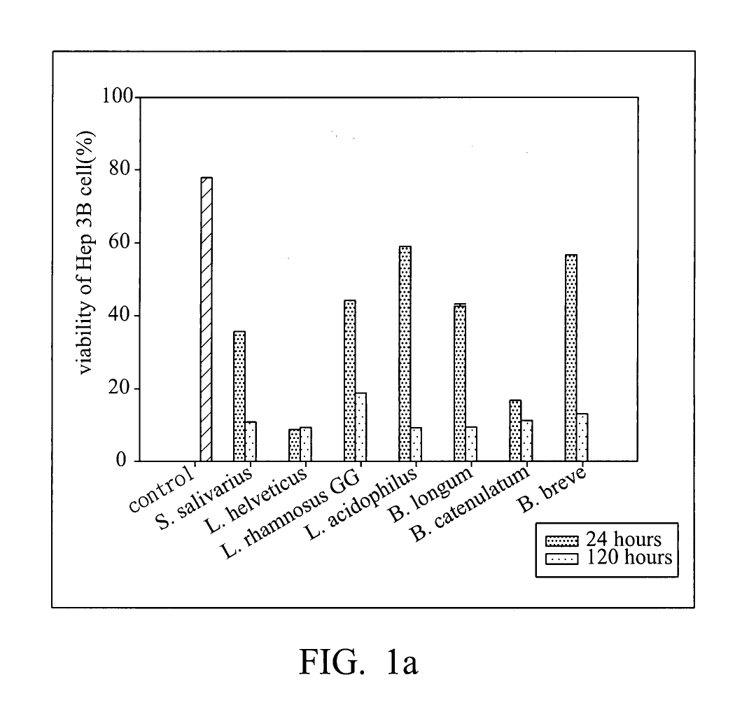

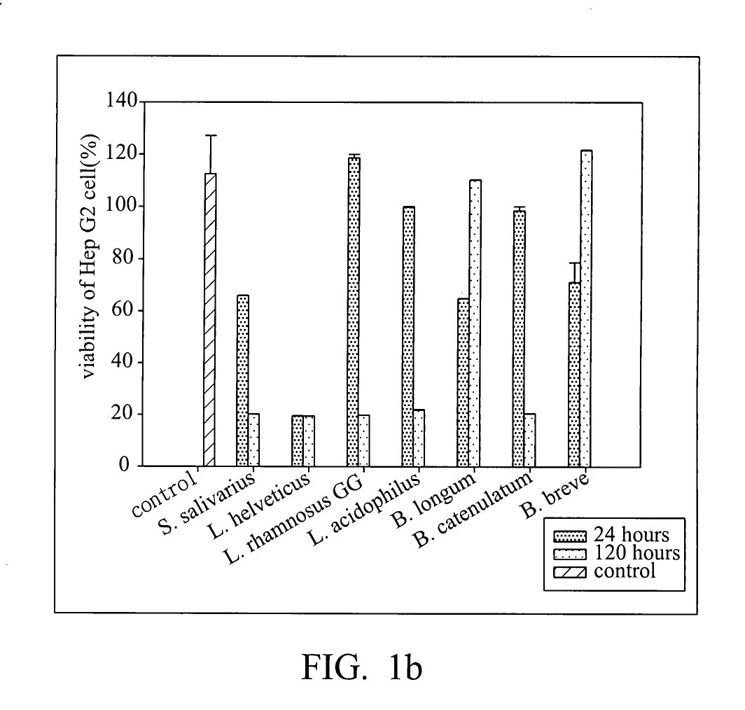

Lactic acid bacteria were added to the Panax notoginseng medium of Example 1, wherein the lactic acid bacteria included Streptococcus salivarius subsp. thermophilus (BCRC12268), Lactobacillus helveticus (BCRC14092, Lactobacillus rhamnosus GG.(BCRC16000), Lactobacillus acidophilus (BCRC10695), Bifidobacterium longum (BCRC14602), Bifidobacterium catenulatum (BCRC14667), and Bifidobacterium breve (BCRC11846), respectively. The initial concentration of the lactic acid bacteria was 104 cfu/ml. The lactic acid bacteria were co-cultured in the Panax notoginseng medium at 37° C. for 24 or 120 hours, and then the fermented Panax notoginseng medium was heated to stop the fermenting process by autoclave. After heating process, bacteria were killed, then solid carbon and nitrogen sources were removed by centrifugation to obtain the fermented product of the invention. The effects of the fermented product on Hep 3B and Hep G2 cell growths were analyzed. For the control group, the fermented product was switched to a non-fermented product. Referring to FIGS. 1a-1b, all fermented Panax notoginseng medium inhibited the growth of Hep 3B and Hep G2 cells, preferably, fermented with Lactobacillus helveticus.

### Example 3

### Comparison of Single and Mixed Strains Lactic Acid Bacteria

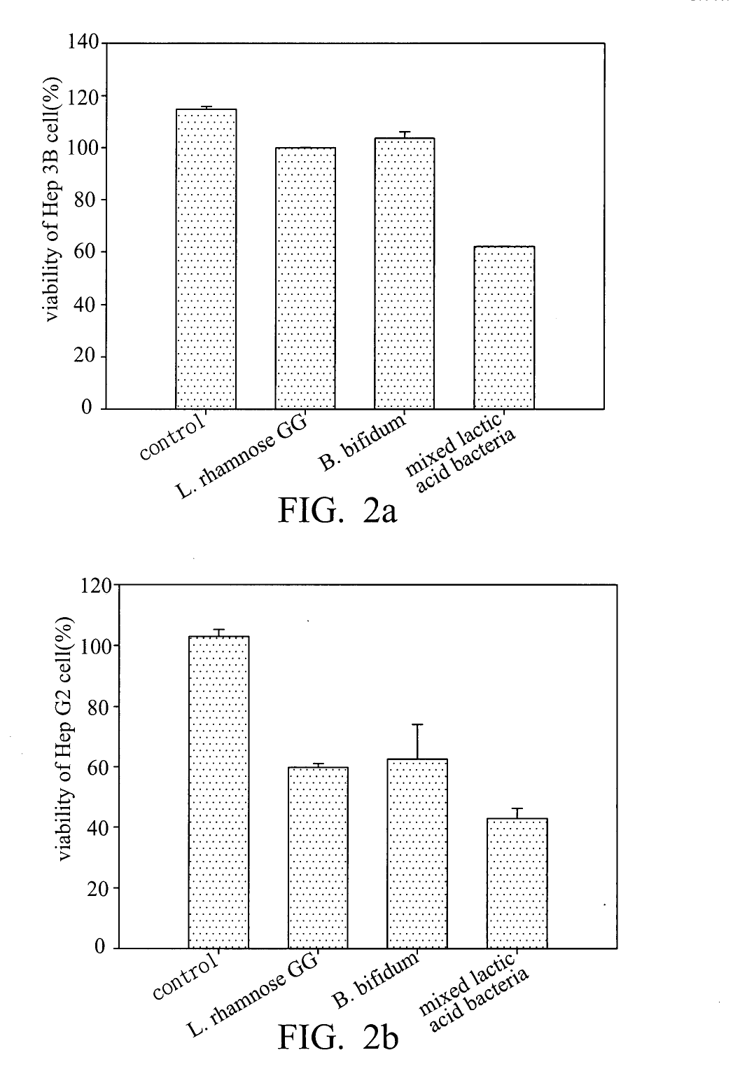

The same procedure carried out in Example 2 was repeated except that the lactic acid bacteria were changed to Bifidobacterium bifidum (BCRC14615) or eight strains mixed lactic acid bacteria (Streptococcus salivarius subsp. thermophilus, Lactobacillus helveticus, Lactobacillus rhamnosus GG, Lactobacillus acidophilus, Bifidobacterium longum, Bifidobacterium catenulatum, and Bifidobacterium breve). The effects of the fermented product on Hep 3B and Hep G2 cell growths were analyzed. FIGS. 2a-2b show that the mixed strains lactic acid bacteria had a better inhibition effect than the single strain of lactic acid bacteria.

### Example 4

### Effect of the Fermentation Temperature on the Inhibition of Cancer Cells Growth

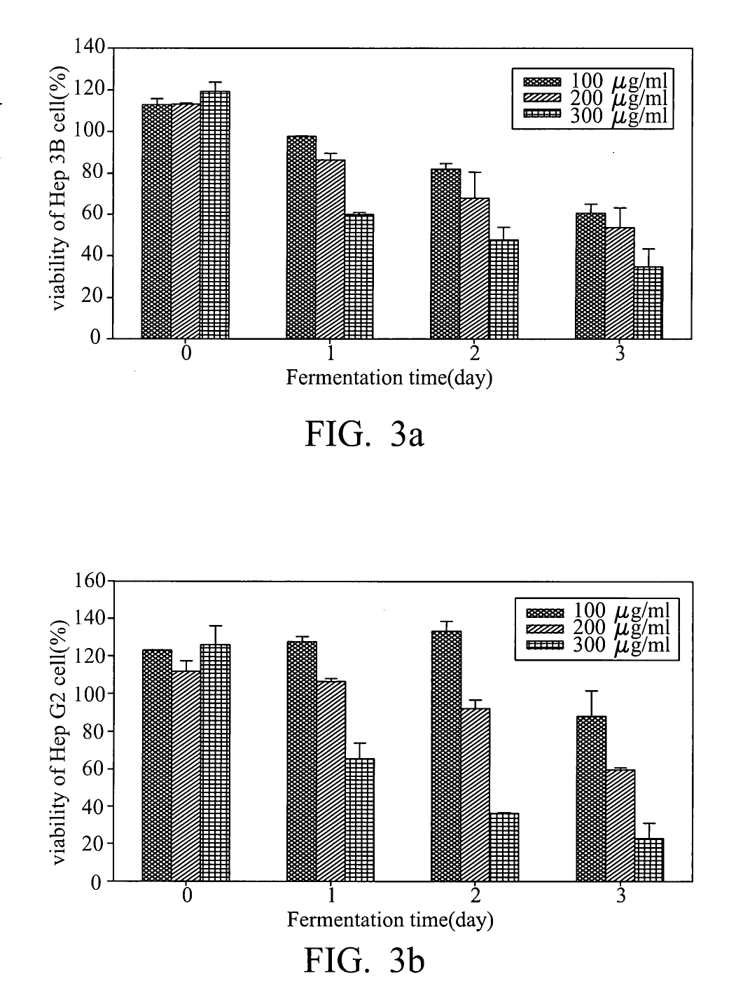

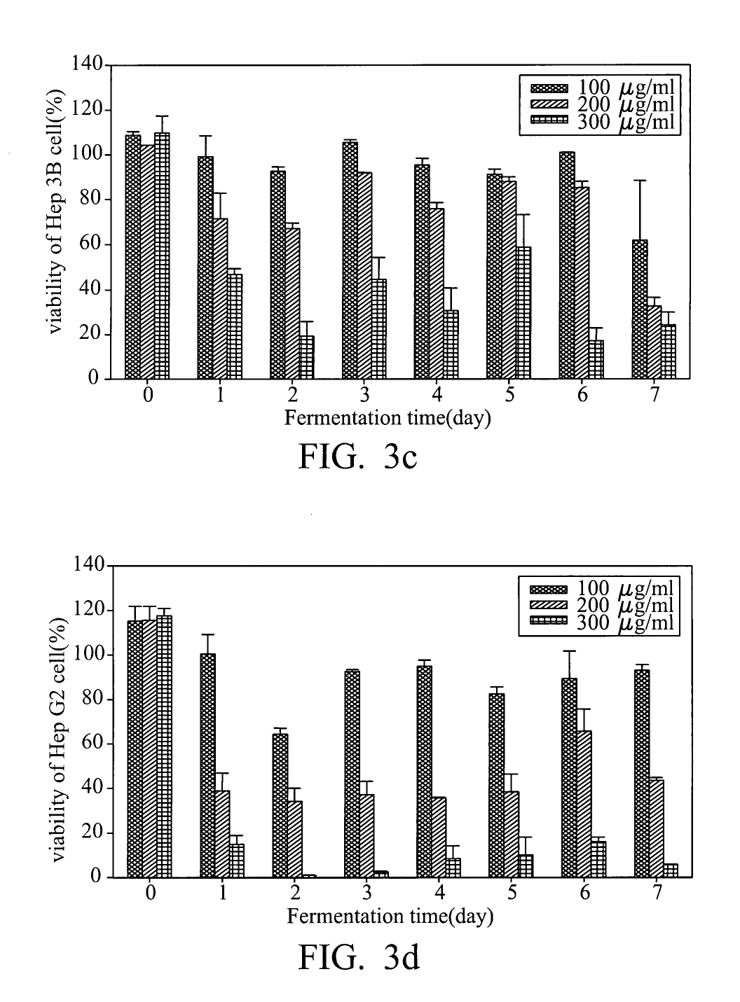

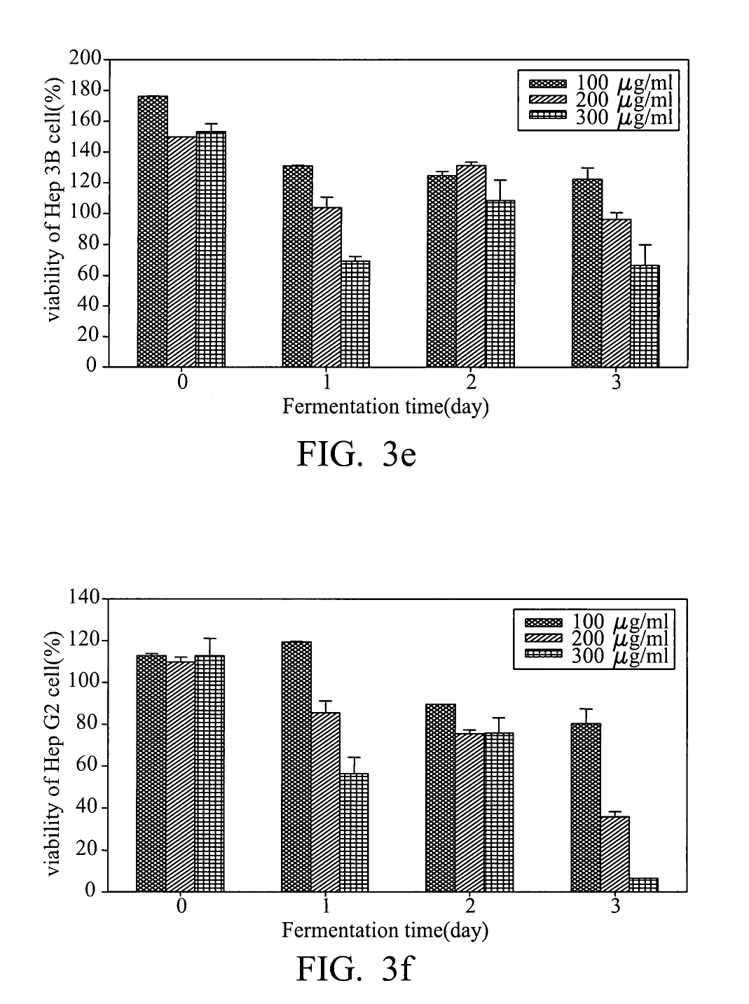

The same procedure carried out in Example 2 was repeated except that the fermentation temperature was changed to 32° C., 37° C. or 42° C., and the concentration of the fermented product included 100, 200, or 300 μg/ml. The capability of repressing Hep 3B and Hep G2 cells by the fermented product was analyzed. Referring to FIGS. 3a-3b, when fermentation temperature was 32° C., the fermented product after 2 days of fermentation had the best inhibition effect. Referring to FIGS. 3c-3d, when fermentation temperature was 37° C., the fermented product after 3 days of fermentation had the best inhibition effect. Referring to FIGS. 3e-3f, when fermentation temperature was 42° C., the fermented product after 3 days of fermentation had the best inhibition effect.

### Example 5

### Effect of pH Value on the Inhibition of Cancer Cells Growth

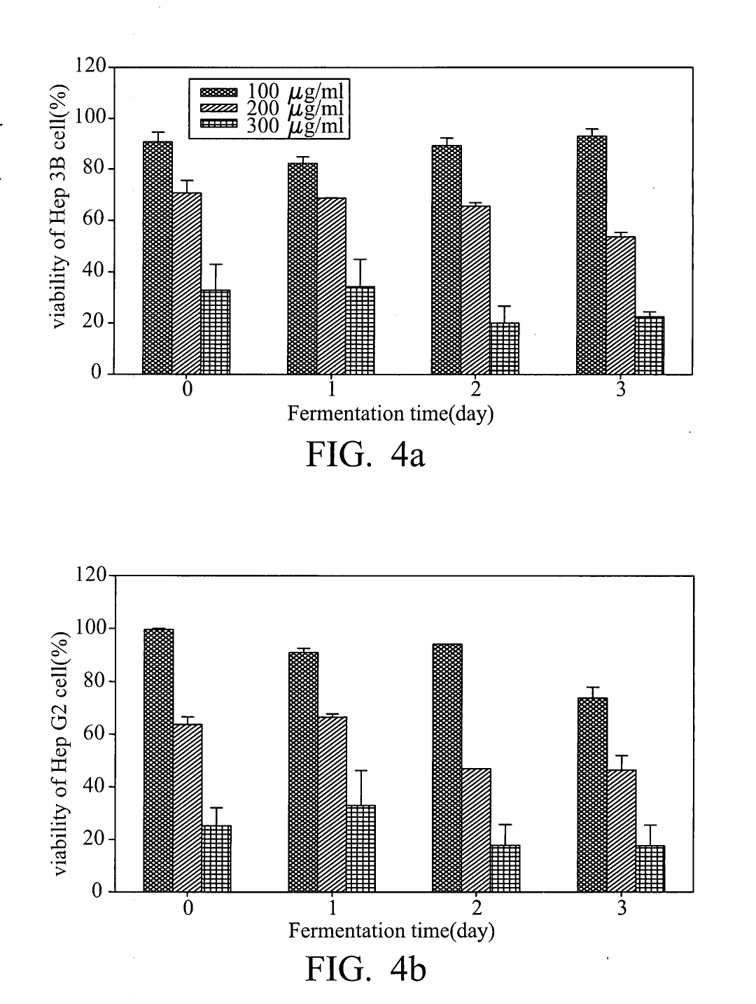

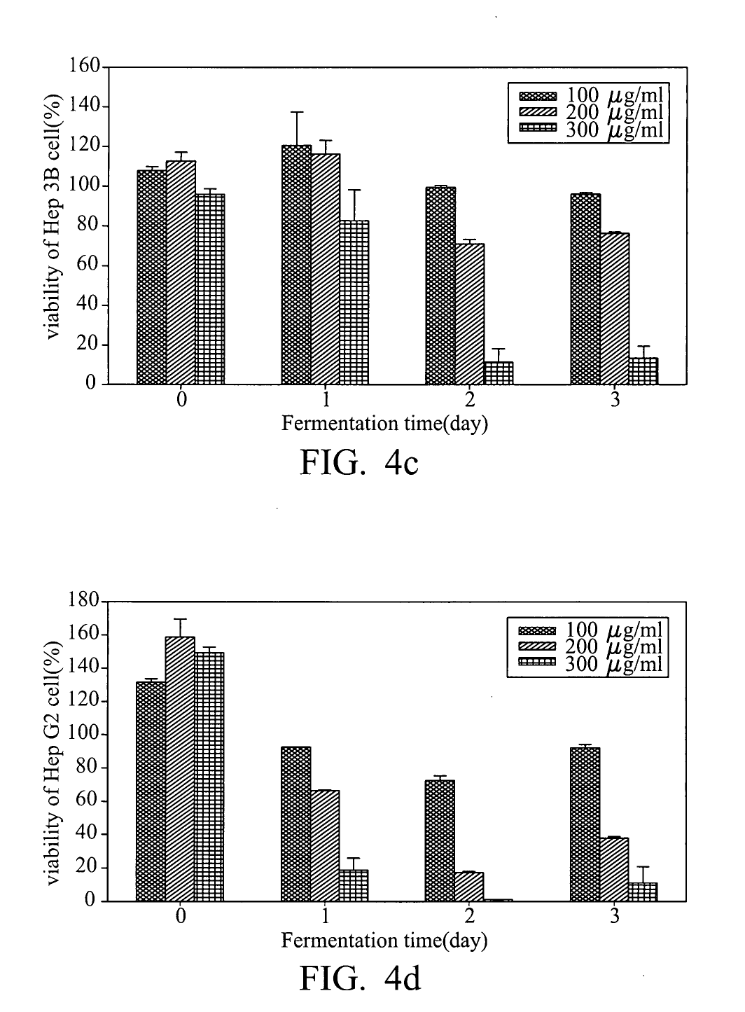

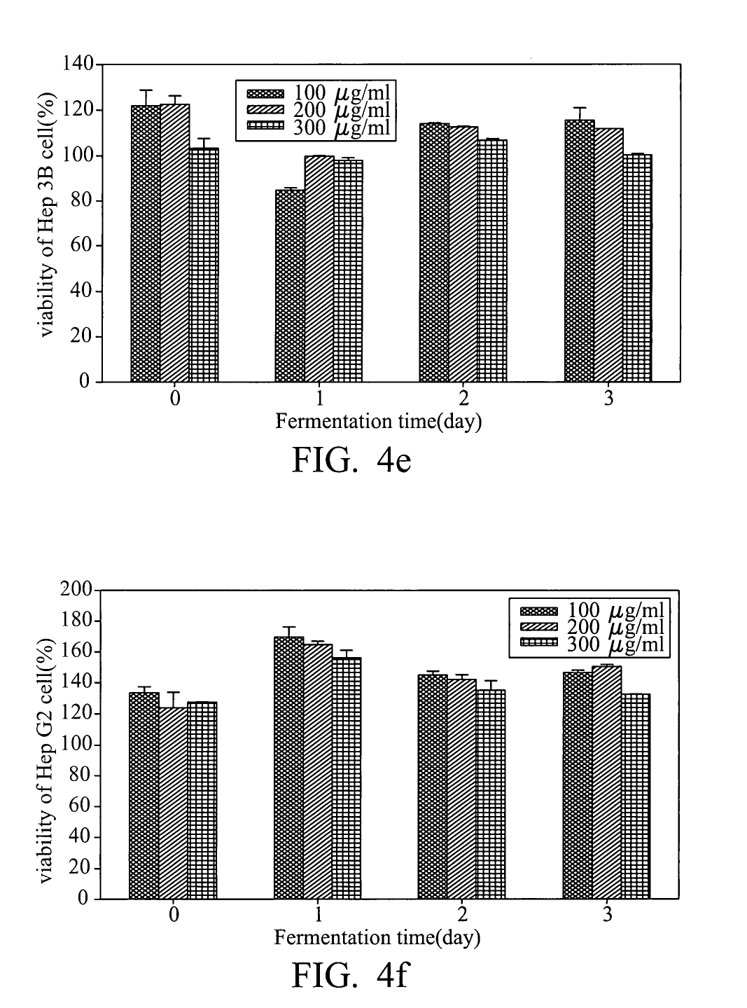

The same procedure carried out in Example 2 was repeated except that the pH value of fermentation was changed to pH 3.5, pH 4.5, or pH 5.5, and the concentration of the fermented product included 100, 200, or 300 μg/ml. The capability of repressing Hep 3B and Hep G2 cells by the fermented product was analyzed. Referring to FIGS. 4a-4d, when the pH value of the fermentation was pH 3.5 or pH 4.5, the fermentation time was preferable to 2 days. Referring to FIGS. 4e-4f, when the pH value of fermentation was 5.5, the fermented product could not inhibit cancer cells.

### Example 6

### Additional Black Bean can Induce the Ability of Anti-Oxidation

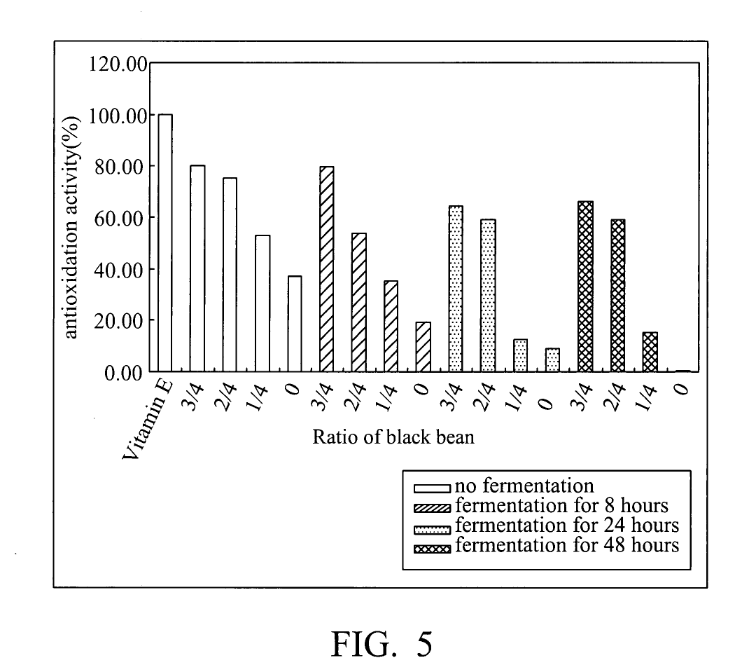

The same procedure carried out in Example 2 was repeated except that the content of the Panax notoginseng medium was changed. The content of the Panax notoginseng medium included 4 types: ¼ Panax notoginseng powder and ¾ black powder, ½ Panax notoginseng powder and 1/2 black powder, ¾ Panax notoginseng powder and ¼ black powder, and all Panax notoginseng powder. In the control group, the fermented product was changed to vitamin E, and the anti-oxidation capacity of vitamin E was defined to 100. Referring to FIG. 5, increasing of the quantity of black bean will result in an increase in the anti-oxidation capacity of the fermented product.

### Example 7

### Effect of the Pre-Heating of Panax notoginseng Medium on the Inhibition of Cancer Cells Growth

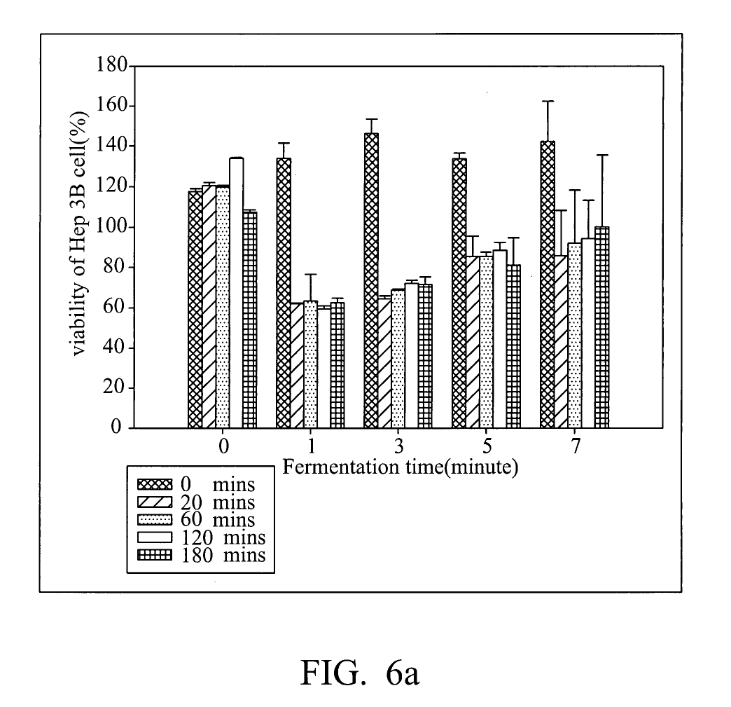

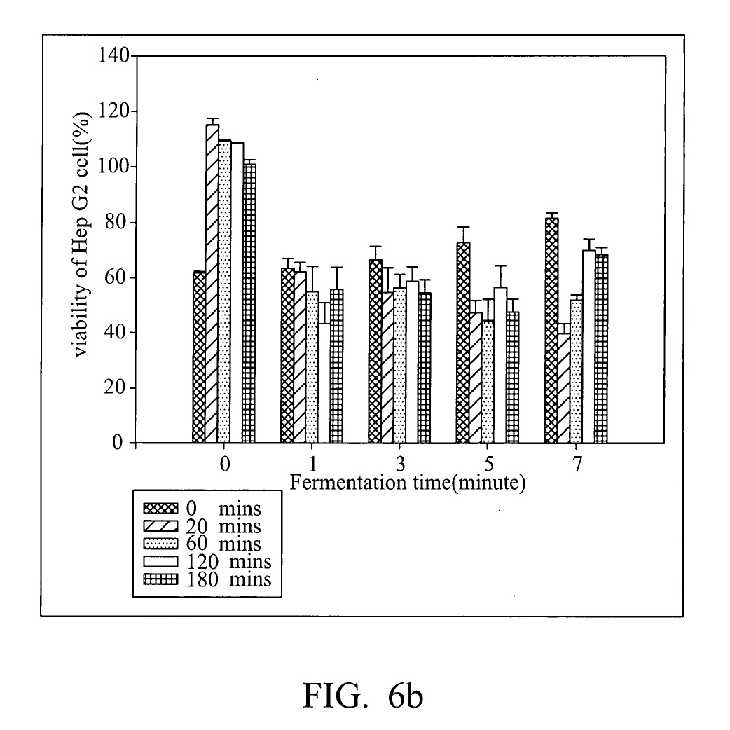

The same procedure carried out in Example 2 was repeated except that the Panax notoginseng medium was pre-heated before fermentation. The conditions of pre-heating were 121° C. for 20, 60, 120, and 180 min, respectively, and the capability of repressing Hep 3B and Hep G2 cells by the fermented product was analyzed. In the control group, the step of pre-heating was not carried out before fermentation. Referring to FIGS. 6a-6b, pre-heating reduced the anti-cancer ability of the fermented product.

### Example 8

### Effect of the Fermented Product on Normal Liver Cells

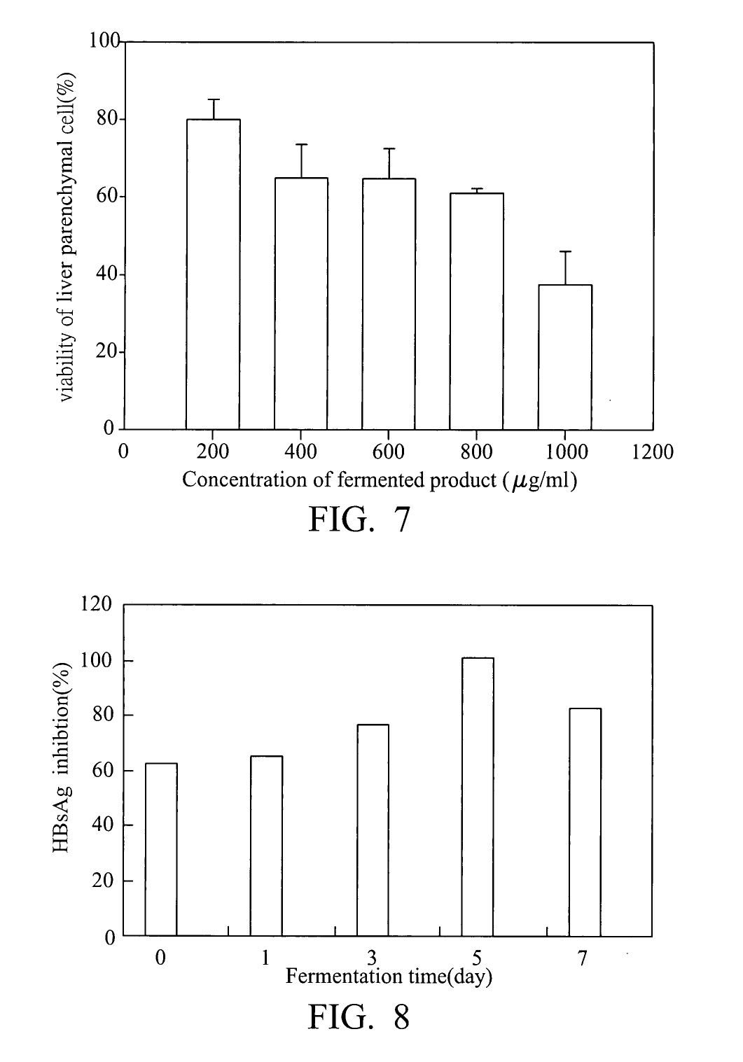

The same procedure carried out in Example 2 was repeated except that cancer cells were changed to mouse primary hepatocyte. The mouse primary hepatocyte was treated with the fermented product of the invention, and then the viability of the mouse primary hepatocytes was detected by a MTT assay. Referring to FIG. 7, when the concentration of the fermented product was 200 μg/ml, the viability of the mouse primary hepatocyte exceeded 80%, and LD50 was 976 μg/ml.

### Example 9

### Effect of the Fermented Product on HBsAG

The same procedure carried out in Example 2 was repeated. Hep 3B cells having HBsAg were treated with the fermented product of the invention fermented for 1, 3, 5, or 7 days, and the inhibition of the HBsAg of the Hep 3B cells was analyzed. Referring to FIG. 8, the inhibition effect of Hep 3B cells by the fermented product increased as the fermentation time increased, and the inhibition rate exceeded 80% when fermentation time was 7 days.

### Example 10

### Animal Experiment

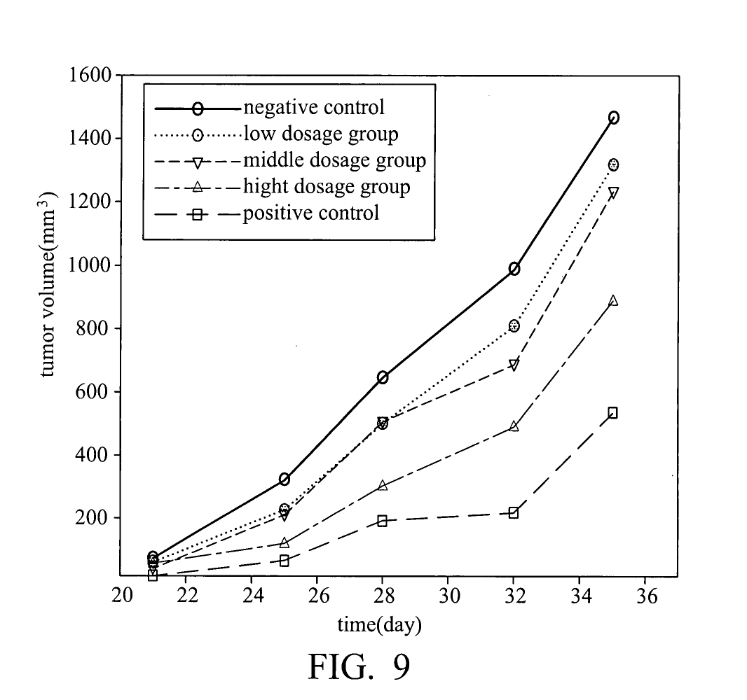

SCID mice with 18 to 22 g body weight were classified into 5 groups and 10 mice per group. First, SCID mice were injected with Hep 3B/T2 cancer cells, and then the SCID mice were fed with different dosages of the fermented product for 35 consecutive days. The tumor size of the SCID mice was measured every 3 to 4 days, and the weight of the heart, liver, spleen, lung, and kidney of the SCID mice was measured when the experiment was finished. The manufacture conditions of the fermented product were 10 wt % of Panax notoginseng powder and 90 wt % of water, incubated at 37° C. for 48 hours with mixed lactic acid bacteria. The lactic acid bacteria included Streptococcus salivarius subsp. thermophilus (BCRC12268), Lactobacillus helveticus (BCRC14092, Lactobacillus rhamnosus GG.(BCRC16000), Lactobacillus acidophilus (BCRC10695), Bifidobacterium longum (BCRC14602), Bifidobacterium catenulatum (BCRC14667), Bifidobacterium breve (BCRC11846), and Bifidobacterium bifidum (BCRC14615). The dosage of the fermented product was classified into a high dosage (1000 μg/ml), a middle dosage (400 μg/ml), and a low dosage (200 μg/ml). In the positive control group, the fermented product was switched to a 5-FU(fluorouracil) drug, in the negative control group, the fermented product was switched to a phosphate buffer, and in the control group, no Hep 3B/T2 cancer cells were injected. Referring to FIG. 9 and Table 1, the tumor in treatment groups showed suppressive effects on cancer cell growth and displayed a dose-dependent relationship.

TABLE 1

| | Control | Negative control | Low dosage |
|---|---|---|---|
| Heart weight (mg) | 149.0 ± 19.7 | 116.8 ± 17.8 | 111.8 ± 9.5 |
| Liver weight (mg) | 1355 ± 123.4 | 1114.5 ± 143.4 | 1160.5 ± 130.6 |
| Spleen weight (mg) | 100.2 ± 12.4 | 59.1 ± 11.7 | 60.4 ± 3.3 |
| Lung weight (mg) | 190.6 ± 8.4 | 178.8 ± 14.2 | 178.5 ± 31.2 |
| Kidney weight (mg) | 411.4 ± 13.3 | 277.5 ± 45 | 321.3 ± 45.5 |
| Tumor weight (mg) | — | 1316.2 ± 403.3 | 1169.9 ± 310.9 |
| | Middle dosage | High dosage | Positive dosage |
| Heart weight (mg) | 107.9 ± 5.5 | 122.7 ± 11.9 | 143.2 ± 14.6 |
| Liver weight (mg) | 1211.5 ± 130.6 | 1110.6 ± 65.7 | 1171.3 ± 103.0 |
| Spleen weight (mg) | 60.4 ± 11.7 | 66.3 ± 8.3 | 118.7 ± 35.6 |
| Lung weight (mg) | 185.3 ± 13.6 | 191.9 ± 19.5 | 215.8 ± 23 |
| Kidney weight (mg) | 325.2 ± 45.3 | 282.2 ± 22.6 | 328.2 ± 26.2 |
| Tumor weight (mg) | 1084.2 ± 344.3 | 537.2 ± 391.9 | 376.1 ± 232.9 |

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for fermenting Panax notoginseng, comprising activating lactic acid bacteria by a culture medium, fermenting a Panax notoginseng medium with the lactic acid bacteria to form a fermented solution, and centrifuging the fermented solution to obtain a supernatant.

2. The method as claimed in claim 1, further comprising adding a black bean extract to the Panax notoginseng medium.

3. The method as claimed in claim 1, further comprising pre-heating the Panax notoginseng medium.

4. The method as claimed in claim 1, wherein the pre-heating is carried out at a temperature of above 75° C.

5. The method as claimed in claim 1, wherein the time of pre-heating exceeds 10 minutes.

6. The method as claimed in claim 1, wherein the lactic acid bacteria comprise Streptococcus salivarius subsp. thermophilus (BCRC12268), Lactobacillus helveticus (BCRC14092, Lactobacillus rhamnosus GG (BCRC16000), Lactobacillus acidophilus (BCRC10695), Bifidobacterium longum (BCRC14602), Bifidobacterium catenulatum (BCRC14667), Bifidobacterium breve (BCRC11846), Bifidobacterium bifidum (BCRC14615), or combinations thereof.

7. The method as claimed in claim 1, wherein the medium is MRS or peptone-yeast extract base medium.

8. The method as claimed in claim 1, wherein the Panax notoginseng medium comprises 10 to 15 wt % of Panax notoginseng powder and 85 to 90 wt % of water.

9. The method as claimed in claim 1, wherein the fermentation time is between 1 and 7 days.

10. The method as claimed in claim 1, wherein the fermentation temperature is between 25° C. and 40° C.

11. The method as claimed in claim 1, wherein pH value of the fermentation is controlled at about 3 to 5.

12. A fermented Panax notoginseng solution prepared by the method of claim 1, wherein the fermented Panax notoginseng solution suppresses the growth of cancer cells.

13. The fermented Panax notoginseng solution as claimed in claim 12, wherein the fermented Panax notoginseng solution inhibits hepatitis B virus surface antigen (HBsAg).

14. The fermented Panax notoginseng solution as claimed in claim 12, wherein the cancer cells comprises the cells of breast cancer, prostate cancer, blood cancer, colorectal cancer, uterine cancer, ovarian cancer, endometrial cancer, cervical cancer, testicular cancer, malignant lymphoma, rhabdomyosarcoma, neuroblastoma, pancreatic cancer, lung cancer, brain cancer, skin cancer, gastric cancer, liver cancer, kidney cancer, or nasopharyngeal cancer.

15. The fermented Panax notoginseng solution as claimed in claim 12, wherein the cancer cell is a liver cancer cell.

16. The fermented Panax notoginseng solution as claimed in claim 12, wherein inhibition of cancer cells by the fermented Panax notoginseng solution exceeds 60%.

17. A composition of preventing and/or treating cancer, comprising an effective amount of fermented Panax notoginseng solution prepared by the method of claim 1, and a pharmaceutically acceptable carrier, or excipient.

18. The composition as claimed in claim 17, wherein the cancer comprises breast cancer, prostate cancer, blood cancer, colorectal cancer, uterine cancer, ovarian cancer, endometrial cancer, cervical cancer, testicular cancer, malignant lymphoma, rhabdomyosarcoma, neuroblastoma, pancreatic cancer, lung cancer, brain cancer, skin cancer, gastric cancer, liver cancer, kidney cancer, or nasopharyngeal cancer.

19. The composition as claimed in claim 17, wherein the cancer is liver cancer.

20. The composition as claimed in claim 17, wherein inhibition of cancers by the fermented Panax notoginseng composition exceeds 60%.

21. The composition as claimed in claim 17, wherein the composition is administrated orally or by injection.
